(19) United States
(12) Patent Application Publication
(10) Pub. No.: US 2010/0211173 A1
(43) Pub. Date: Aug. 19, 2010

(54) ARTICULAR CARTILAGE, DEVICE AND METHOD FOR REPAIRING CARTILAGE DEFECTS

(76) Inventors: Tamás Bárdos, Kaposvar (HU); Ârpád Bellyei, Pecs (HU); Tamás Illés, Pecs (HU); Péter Németh, Pecs (HU)

Correspondence Address: HAHN & VOIGHT PLLC, 1012 14TH STREET, NW, SUITE 620, WASHINGTON, DC, 20005, US

(21) Appl. No.: 12/674,772

(22) PCT Filed: Aug. 1, 2008
(86) PCT No.: PCT/HU08/00095

§ 371 Date: Mar. 11, 2010

(30) Foreign Application Priority Data
Aug. 10, 2007 (HU) P0700524

(51) Int. Cl. A61F 2/08; A61B 17/32

(52) U.S. Cl. 623/13.11; 606/79

(57) ABSTRACT

The articular cartilage according to the invention is made of pure cartilage and is provided with incisions (12) on the surface facing the bone. The cartilage cells are preferably seeded on the surface provided with incisions (12). The method for producing the articular cartilage comprises the step of collecting cartilage from joints, wherein pure cartilage is collected without bone, and incisions are made on the surface of the cartilage intended to face the bone. It is preferably fresh frozen until use. The device for harvesting articular cartilage, comprises handle and cutting blade, wherein the cutting blade (4) is curvilinear and is provided with spacer elements (5), meanwhile the device for producing incisions in articular cartilages comprises handle (2) and a bridge (3) connected to said handle (2) and being provided with one or more cutting blade(s) (4). During the method for applying the articular cartilage the articular cartilage is fixed by thin surgical yarn stitches, by fibrin glue or by small anchors (FIG. 8).

### FIELD OF THE DISCLOSURE

The present invention relates to articular cartilages for repairing cartilage defects and a method for producing articular cartilage comprising the step of collecting cartilage from joints. Further object of the invention is a device for harvesting articular cartilage, comprising handle and cutting edge as well as another device for producing incisions in articular cartilages.

### BACKGROUND OF THE INVENTION

Joint cartilage defects or deceases can result in progressive impairment of life quality. The so called biological regeneration methods are more and more applied worldwide, besides the protetical reconstructions. One of these methods is tissue engineering, advancing continuously. Tissue engineering offers a wide field of applications in clinical work, and it seems that this method will be the revolutionary technology for healing, further to the gene-technology. The majority of people above 65 years have joint defects due to the decreased ability of regeneration (primary osteoarthrosis) or to the increased load (secundary osteoarthrosis) of the cartilage tissues. All these cartilage defects are still curable in initial stage. However, the simple biological reparation methods available for the time being (abrasion, drilling, debridement, shaving or microfracture) proved in long term examinations to be insufficient, as the produced fibrous cartilage is mechanically weak.

Recently, mosaic-plasty and autologous cell transplantation have been developed as modern cartilage replacement technologies.

In the case of mosaic-plasty, bone based bone-cartilage coloumns of 4-8 mm diameter are taken from non-weight-bearing surface of the knee joint of the patient, and grafts are implanted to the affected area of the same knee joint (Hangody L, Rathonyi G K, Duska Z, Vasarhelyi G, Fules P, and Modis L. 2004. Autologous osteochondral mosaicplasty. Surgical technique. J Bone Joint Sung Am 65-72.). U.S. Pat. No. 6,241,756 or U.S. Pat. No. 6,358,253 disclose similar methods. Such osteochondral (bone based cartilage graft) replacements may be sufficient for reparing smaller (<4 cm²) defects, but medium or greater surfaces can not be treated in this way, as the amount of donor regions of the knee joint are restricted. A further problem is that the integrity of the subchondral bone is broken during the preparation.

The method of autologous chondrocyte implantation (ACI) advanced quickly since the first publication (Brittberg M, Lindahl A, Ohlsson C, Isaksson O, and Peterson L. 1994. Treatment of deep cartilage defects in the knee with autologous chondrocyte transplantation. N Engl J. Med. 889-895.). It is almost an everyday practice in the US and Western-Europe to gather cartilage cells from donor area, to culture them in a laboratory specified to this work and to implant them back to the damaged joint cartilage. The number of ACI operations exceeds 20 000. In case of first generation ACI (developed first), cells cultured for 20-50 days are reimplanted in cell suspension, without supporting matrix, by injecting them below a graft stitched to the cartilage. In case of second generation ACI, the cells grown in the laboratory are seeded onto a supporting matrix (collagen filaments or artificial degradable polymers), and only the final graft should be secured to the defected cartilage area.

These methods are already applicable for replacing greater defects (up to 10 cm²), however, the structure of the tissue is not the preferred hyaline-cartilage structure, i.e. the orientation of the collagen filaments does not show the original cartilage structure.

Object of the present invention is therefore to provide a solution to eliminate the problems outlined above.

### SUMMARY OF THE INVENTION

According to the invention articular cartilages are provided, which are made of pure cartilage and have incisions on the surface facing the bone.

The distance between the incisions may be of 0.1-1 mm, and the incisions are parallel with each other or are of different directions. They preferably have a depth leaving an intact layer of at least 50 μm thickness.

According to a preferred embodiment, cartilage cells, first of all hyaline cells taken from joint cartilages are seeded on the surface provided with incisions.

The method according to the invention comprises the step of collecting cartilage from joints, wherein pure cartilage is collected without bone, and incisions are made on the surface of the cartilage intended to face the bone and a distance of 0.1-1 mm is left between the incisions, meanwhile an intact layer of at least 50 μm thickness is left at the outer side of the cartilage.

According to the method, cartilage cells, preferably hyaline cells taken from joint cartilages are seeded on the surface intended to face the bone. It may be advantageous if the articular cartilage is fresh frozen until use.

For harvesting articular cartilage, a device may be applied comprising handle and cutting blade, wherein the cutting blade is curvilinear and is provided with spacer elements.

The distance between the cutting blade and the spacer elements is preferably 0.1-4 mm and the curvature of the edge is adjusted to that of the joint surface.

The device for producing incisions in the articular cartilages comprises a handle and a bridge connected to said handle and being provided with one or more cutting blade(s). The thickness of the cutting blades is 0.1-0.5 mm, and the distance between the cutting blades is 0.1-1 mm.

The cutting blades may be arranged on discs or on plates and may be provided with adjustable spacer elements.

During the method for applying the articular cartilage—if it is not seeded with cells—microfracturing is performed first at the cartilage defect and than the articular cartilage is fixed. If the articular cartilage is provided with cartilage cells, it is directly fixed at the cartilage defect.

The articular cartilage may be fixed by thin surgical yarn stitches or fibrin glue. It is also possible that the articular cartilage is fixed by small pieces of surgical yarn or small anchors introduced through the bone.

The invention is based on the recognition that thin cartilage allografts of great surfaces are optimal for the replacement of defected joint cartilages, and the efficiency of their use may be improved if the side intended to face the bone is provided with incisions, and preferably with cartilage cells as well. It is also recognized, that these cells transplanted into the matrix have the optimal structure if they are applied to a matrix gathered from a cadaver and cleared, preferably completely, from cells. A device for harvesting articular cartilage and another device for producing incisions in articular cartilages have been developed for this purpose.

### BRIEF DESCRIPTION OF THE DRAWING

Further details of the invention will be set forth below in conjunction with the drawing where

FIG. 1. is a schematic view of a first embodiment of the device according to the invention for harvesting articular cartilage,

FIG. 2. is a schematic view of a second embodiment of the device for harvesting articular cartilage,

FIG. 3. shows the steps of harvesting sterile articular cartilage,

FIG. 4. is a schematic view of a first embodiment of the device according to the invention for producing incisions in articular cartilages,

FIG. 5. is a schematic view of a second embodiment of the device for producing incisions in articular cartilage,

FIG. 6. is an enlarged top view of a preferred embodiment of an articular cartilage according to the invention,

FIG. 7. is section VII-VII of FIG. 6,

FIG. 8. is the cross section of an articular cartilage provided with cartilage cells, after implanting and

FIG. 9. is the cross section of an articular cartilage without transplanted cells, after implanting.

### DESCRIPTION OF THE PREFERRED EMBODIMENTS

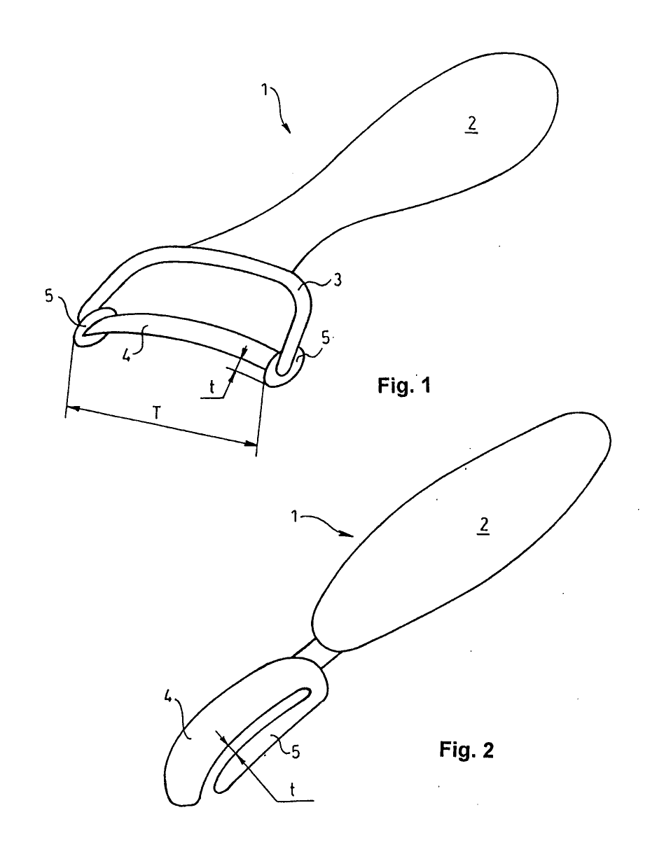

Turning to FIG. 1, a device for harvesting articular cartilage 1 comprises a handle 2 provided with a sharp, curvilinear cutting blade 4 fixed in a bridge 3. The curvature of the cutting blade 4 is adjusted to that of the joint surface to be harvested. At the ends of the cutting blade 4, there are spacer elements 5. The distance t between the edges of the spacer elements 5 and the edge of the cutting blade 4 defines the depth of harvesting, i.e. the distance from the bone/cartilage border (tidemark). This distance is in this case 0.5 mm. The distance T between the edges of the spacer elements 5 defines the width of the harvesting.

FIG. 2. illustrates another embodiment of the device for harvesting articular cartilage 1 according to the invention. This device also comprises a handle 2 with a sharp, curvilinear cutting blade 4 fixed thereon. Blade 4 is provided with a spacer element 5, too. This element is in this embodiment a support plate. The distance t between the edge of the spacer element 5 and the edge of the blade 4 is in this case 0.5 mm, but can go up to 4 mm, if needed.

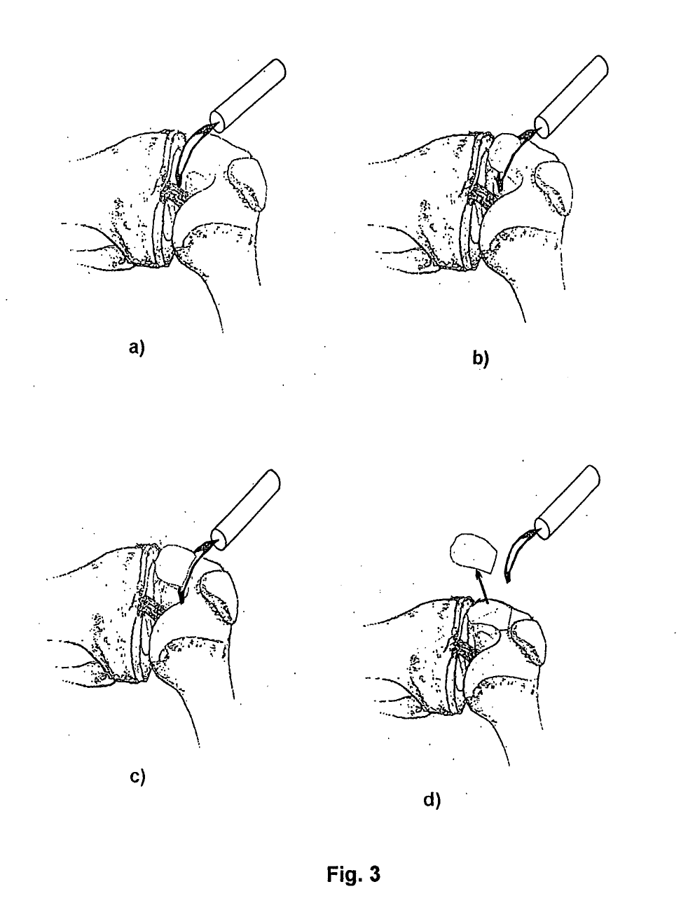

The thickness of the blade 4 of the device 1 according to the invention for harvesting articular cartilage ranges preferably from 0.1 to 0.5 mm, and cartilages of rather big surfaces (6-10 cm²) can be harvested therewith. The steps of harvesting are shown in FIGS. 3a-3d.

Before implantation, the harvested articular cartilage should be provided with incisions according to the invention, said incisions providing an indentation on the side of the cartilage facing the bone. The distances between the incisions should be very small: 0.1-1 mm. A device 6 for producing such incisions is shown in FIG. 4 (the illustration is schematic and the proportions are not real). The device 6 comprises a handle 2 provided with a bridge 3 on one end, and cutting blades 4 arranged in the bridge. The thickness of the cutting blades 4 is 0.2 mm according to this embodiment, and the distances between them is 0.4 mm.

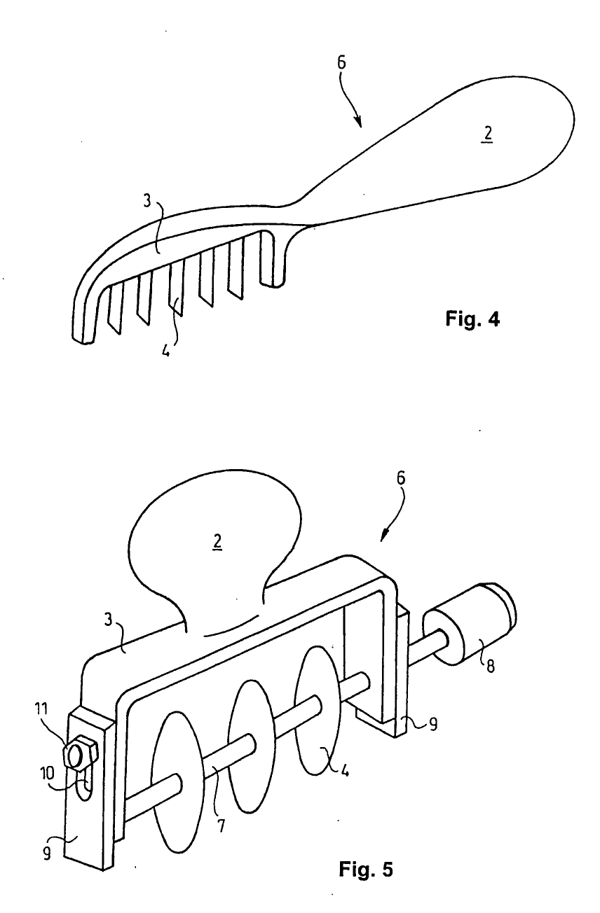

FIG. 5. shows another embodiment of the device 6 for producing incisions (the illustration is schematic and not scaled). Here, the cutting blades 4 in the bridge 3 are discs arranged on a rod 7. The discs are fixed (in other embodiments they may be arranged rotatably) on the rod and the rod is provided with a drive 8 (preferably an electric motor). The depth of the cuts can be adjusted by legs 9 slidably arranged on the bridge 3. The legs can be fixed at the desired height with slots 10 and nuts 11.

Other embodiments of the device 6 for producing incisions may be applied as well. One of them may resemble to an egg cutter device: it may have a base and then the handle 2 provided with a bridge 3 on one end, and cutting blades 4 arranged in the bridge is formed as a cutting arm tiltably connected to said base. The depth of the incisions can be adjusted by changing the position of the cutting blades 4 with respect to the legs 9 of the bridge 3.

For preparing the incisions, cutting arm is opened, a cartilage is arranged on the upper surface of the base and then the cutting arm is turned down, until legs butt on base.

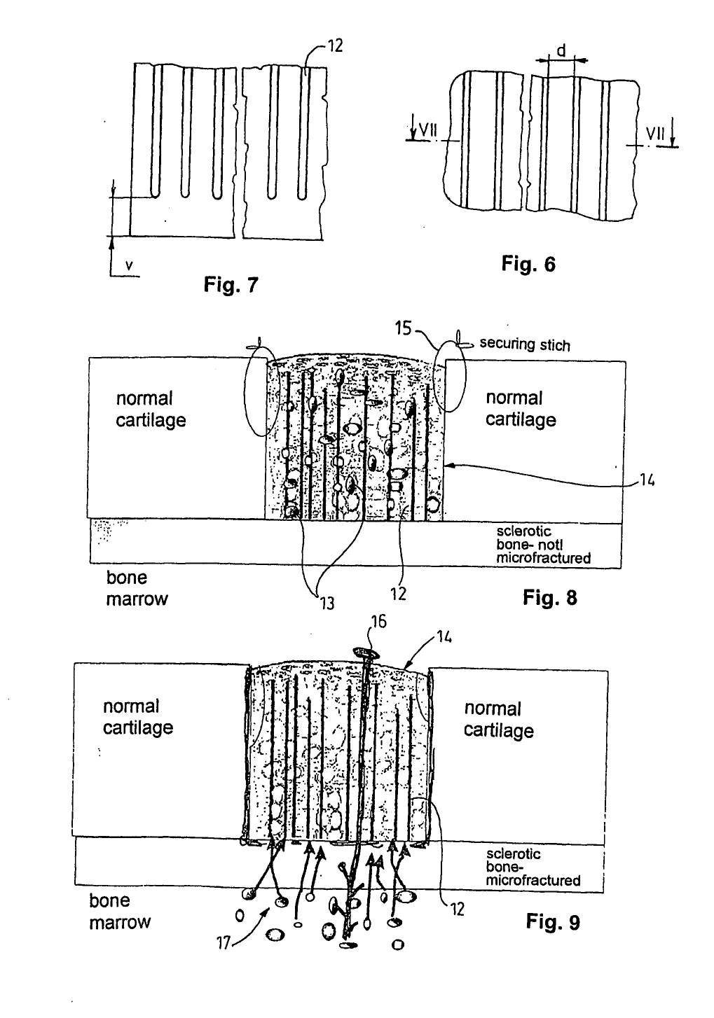

An articular cartilage obtained in the above way is illustrated in FIGS. 6 and 7, wherein FIG. 6. is a top view and FIG. 7. is a cross section of the cartilage. Incisions 12 produced with one embodiment of device 6 have a depth to leave an intact layer of cartilage. The minimum thickness v of that layer is 50 μm, but may go up to 1000 μm. The value of v for the embodiment shown in FIGS. 7 and 8 is 100 μm. The incisions 12 are parallel with each other, but any other pattern may be used. The distances d between the incisions 12 may range from 0.1 to 1 mm, it is 0.6 mm for the embodiment shown in FIGS. 6 and 7.

### EXAMPLES

### Example 1

Several hundred milligrams of hyaline cartilage was collected with arthroscopy for repairing the cartilage damage of a young sportsman. The collected cartilage was delivered to a cell culturing laboratory.

After having obtained the required number of cells, they were suspended, poured onto the side of the matrix provided with incision, and left for properly sedimenting.

The cartilage matrix had been harvested in sterile conditions from the knee joint of a cadaver, long before the operation, with the device shown in FIG. 1. The matrix with a surface of 2×3 cm had been provided with incisions on the side facing the bone, with the device shown in FIG. 5. The incisions had been made in two perpendicular directions, wherein the distances between the incisions were 0.5 mm and the thickness of the intact collagen layer was 90 μm. The matrix had than been provided with a sterile packing and stored on a temperature of −80° C.

The cartilage matrix obtained from a cadaver and prepared in the above outlined way was implanted via miniarthrotomy knee operation, as shown in FIG. 9. In the exposed knee joint, the damaged cartilage part was removed with a sharp spoon, up to the intact cartilage and a quadratic recipient cavity was prepared in the cleared surface. The graft 14 provided with cells 13 was cut to fit in the cavity and implanted in the appropriate position. It was then connected to the edge of the intact cartilage layer with small stitches. At last, the implant was glued around (sealing) with fibrin glue.

### Example 2

A patient of middle age had ankle complaints. As the result of an examination, it was found that he had focal cartilage defect on the upper surface of her talus. It was decided to perform cartilage substitution by cartilage cell transplantation, therefore bone marrow stem cells were collected for culturing (in cases, when it is not possible, joint cartilage particles may be collected for obtaining cells). The collected cells were delivered to a cell culturing laboratory.

Prior to the operation, cartilage sample had been harvested in sterile conditions from the knee joint of a cadaver, with the device shown in FIG. 2. The cartilage had been processed with incisions on the side facing the bone, with the device shown in FIG. 5. The incisions had been made in parallel directions, wherein the distances between the incisions were 0.8 mm and the thickness of the intact layer was 120 μm.

The multiplied cells were centrifuged to the graft provided with incisions, to be captured in the incisions and were fixed therein with glue.

The cartilage had than been provided with a sterile packing and stored in fluid nitrogen on a temperature of −160° C. until the day of the operation, when it was sent to the operating room.

After having exposed the ankle joint, the cartilage defect of the talus was cleared, the bone below was cleaned and the graft prepared and cut to proper size and form in advance was implanted in place of the cartilage deficiency. For fixing, fibrin glue was applied, without stitching, on the bottom and the sides of the implant. Thereafter, the joint was covered and a rehabilitation protocol of 6 weeks has been carried out with proper fractional load.

### Example 3

During arthroscopy of a women of middle age it was found that she had small cartilage deficiency on the knee joint. Therefore, at the same time, following a small joint exposure, the region of the cartilage deficiency has been cleared up to the healthy cartilage.

One week before the operation sound articular cartilage had been harvested in sterile conditions from the shoulder joint of a cadaver, with the device shown in FIG. 2. The cartilage had been provided with incisions on the side facing the bone. The incisions had been made in parallel directions, wherein the distances between the incisions were 0.1 mm and the thickness of the intact layer was 500 μm.

The cartilage had than been stored for one week in sterile conditions, without freezing, on +4° C., until the day of the operation.

The graft was delivered to the surgeon together with the living cells therein, for operation. A hole was made in the bone below the cartilage (microfracture) and the graft 14, after having been cut to proper size and form, was fixed in the region of the cartilage deficiency, the surface provided with incisions facing the bone. For fixing the graft, small anchors 16 were introduced through the bone.

In this case, bone marrow cells 17 could flow to the incisions 12 through the hole (not shown) in the bone, and these cells produced the cellular body of the articular cartilage by conversion to cartilage cells. The cells surviving in the cartilage also helped the cartilage to stick to the subchondral bone.

The drawing and the examples show, that the devices according to the invention are simple, the use of them is safe, and they enable to prepare articular cartilages of far better quality, than the ones used up to now.

The articular cartilages according to the invention offer the advantage with respect to the state of art, that the incisions considerably improve the incorporation of the cells cultured in laboratory or deriving from bone marrow. A further advantage is that the incisions enhance the flexibility of the cartilage and, in this way, the use is more simple and safe.

1. Articular cartilage for repairing cartilage defects, characterized in that it is made of pure cartilage and is provided with incisions (12) on the surface facing the bone and the incisions (12) have a depth leaving an intact layer (v) of at least 50 μm thickness.

2. Articular cartilage as claimed in claim 1, characterized in that there is a distance of 0.1-1 mm between the incisions (12).

3. (canceled)

4. (canceled)

5. (canceled)

6. Articular cartilage as claimed in claim 1, characterized in that cartilage cells are seeded on the surface provided with incisions (12).

7. Articular cartilage as claimed in claim 6, characterized in that the cartilage cells are hyaline cells taken from joint cartilages.

8. Method for producing articular cartilage for repairing cartilage defects comprising the step of collecting cartilage from joints, characterized in that pure cartilage is collected without bone, and incisions are made on the surface of the cartilage intended to face the bone.

9. The method as claimed in claim 8, characterized in that a distance of 0.1-1 mm is left between the incisions (12).

10. The method as claimed in claim 8, characterized in that an intact layer (v) of at least 50 μm thickness is left at the outer side of the cartilage.

11. The method as claimed in claim 8, characterized in that cartilage cells are seeded on the surface intended to face the bone.

12. The method as claimed in claim 8, characterized in that hyaline cells are taken from joint cartilages for arranging them on the surface intended to face the bone.

13. The method as claimed in claim 8, characterized in that the articular cartilage is fresh frozen until use.

14. (canceled)

15. (canceled)

16. (canceled)

17. Device for producing incisions in articular cartilages, characterized in that it comprises a handle (2) and a bridge (3) connected to said handle (2), and being provided with one or more cutting blade(s) (4) and spacer elements (5), which spacer elements (5) are adjustable supports (9).

18. (canceled)

19. (canceled)

20. (canceled)

21. (canceled)

22. (canceled)

23. (canceled)

24. The device as claimed in claim 17, characterized in that the handle (2) and the bridge (3) connected to said handle (2) are in the form of a cutting arm tiltably connected to a base.

25. Method for applying the articular cartilage as claimed in claim 1, for repairing cartilage defects, characterized in that microfracturing is performed first at the cartilage defect and then the articular cartilage is fixed.

26. Method for applying the articular cartilage as claimed in claim 6, for repairing cartilage defects, characterized in that the articular cartilage provided with cartilage cells is directly fixed at the cartilage defect.

27. The method as claimed in claim 25, characterized in that the articular cartilage is fixed by thin surgical yarn stitches.

28. The method as claimed in claim 25, characterized in that the articular cartilage is fixed by fibrin glue.

29. The method as claimed in claim 25, characterized in that the articular cartilage is fixed by small pieces of surgical yarn introduced through the bone.

30. The method as claimed in claim 25, characterized in that the articular cartilage is fixed by small anchors introduced through the bone.
